(19) United States
(12) Patent Application Publication
(10) Pub. No.: US 2025/0366788 A1
(43) Pub. Date: Dec. 4, 2025

(54) A METHOD FOR CORRECTING A DRIFT EFFECT IN MEASURED DATA OBTAINED USING AN IMPLANTABLE PRESSURE SENSOR

(71) Applicant: BIOTRONIK SE & Co. KG, Berlin (DE)

(72) Inventors: Christian MOSS, Falkensee (DE); Andreas ARNDT, Berlin (DE)

(73) Assignee: BIOTRONIK SE & Co. KG, Berlin (DE)

(21) Appl. No.: 18/707,931

(22) PCT Filed: Oct. 24, 2022
(86) PCT No.: PCT/EP2022/079602

§ 371 Date: May 7, 2024

(30) Foreign Application Priority Data
Nov. 8, 2021 (EP) 21206857.1

(51) Int. Cl. A61B 5/00; A61B 5/0215

(52) U.S. Cl. CPC A61B 5/7203; A61B 5/0031; A61B 5/0215; A61B 2560/0257

(57) ABSTRACT

A method for correcting a drift effect in measured data obtained using an implantable pressure sensor comprises: obtaining processing data, based on measured data indicative of a measured pressure obtained using the implantable pressure sensor; deriving, from said processing data, a multiplicity of fit values; determining, using said fit values, a fitted curve; and correcting said processing data based on the fitted curve to compensate for a drift effect in said measured data obtained using the implantable pressure sensor.

### CROSS-REFERENCE TO RELATED APPLICATIONS

This application is the United States National Phase under 35 U.S.C. § 371 of PCT International Patent Application No. PCT/EP2022/079602, filed on Oct. 24, 2022, which claims the benefit of European Patent Application No. 21206857.1, filed on Nov. 8, 2021, the disclosures of which are hereby incorporated by reference herein in their entireties.

### TECHNICAL FIELD

The instant invention concerns a method for correcting a drift effect in measured data obtained using an implantable pressure sensor, and a system for obtaining data indicative of a pressure in a patient.

### BACKGROUND

An implantable pressure sensor may, for example, be configured for implantation into the pulmonary artery of a patient in order to conduct pressure measurements within the pulmonary artery. A pressure sensor of this kind is implantable in a patient, and is operative to function over a prolonged period of time within the patient, the pressure sensor, for example, being in communication connection with a patient device external to the patient. The patient device may be part of a telemedical monitoring system (home monitoring system) and may be configured to communicate with a telemedical monitoring service center in order to allow for a remote monitoring of vital data of the patient.

Within a conventional setup, a pressure sensor transmits measured data to an external device, which processes the data and transmits the data to a telemedical monitoring service center remote from the patient, at which the data may be reviewed and analyzed for monitoring the patient. The external device herein may process the measured data obtained from the pressure sensor by relating the measured data to a reference value corresponding to an atmospheric pressure value in order to derive pressure information indicative of the (relative) pressure at the site of the pressure sensor within the patient.

Pressure sensors could exhibit a drift in rare cases, which has an effect on a sensor output in that the sensor output relating to a specific pressure at the site of the pressure sensor progressively changes over time, for example due to an aging of the sensor or a strain or stress acting on a housing of the sensor. A drift generally impacts the measured data, hence rendering the measured data increasingly inaccurate over time.

Within regular sensor devices, a drift effect may be compensated for by means of calibration, which is repeated at certain intervals. However, an implantable pressure sensor cannot easily be calibrated, as the implantable pressure sensor is not easily accessible within the patient.

The present disclosure is directed toward overcoming one or more of the above-mentioned problems, though not necessarily limited to embodiments that do.

### SUMMARY

It is an object of the instant invention to provide a method for correcting a drift effect in measured data obtained using an implantable pressure sensor and a system for obtaining data indicative of a pressure in a patient which allow to provide accurate measurement data allowing for an accurate pressure reading within the patient.

At least this object is achieved by means of the method comprising the features of claim 1.

Accordingly, a method for correcting a drift effect in measured data obtained using an implantable pressure sensor comprises: obtaining processing data, based on measurement data indicative of a measured pressure obtained using the implantable pressure sensor: deriving, from said processing data, a multiplicity of fit values: determining, using said fit values, a fitted curve; and correcting said processing data based on the fitted curve to compensate for a drift effect in said measured data obtained using the implantable pressure sensor.

Within the method, processing data are obtained based on measured data as output by the implantable pressure sensor. From the processing data, fit values are derived, which may be based on a filtering of the processing data in order to provide a number of values according to which a fitting may be performed. By means of the fit values a fitted curve is determined, the fitted curve being indicative of a progression within the processing data which potentially may be due to a drift effect in the measured data. Based on the fitted curve, then, the processing data may be corrected in order to obtain corrected data, in which a drift effect is compensated for such that the corrected processing data can be assumed to be substantially drift free.

Pressure data within a patient can generally be assumed to vary over time, wherein the pressure may exhibit positive and negative peaks about a mean value. If a mean value of the measured data is found to progressively change, for example progressively increase, it may be assumed that such progressive change is due to a drift effect.

However, a change in the mean value may not only be due to a drift effect, but may also have a physiological origin, due to a change in condition of the patient. Hence, a correction of the measured data should not simply be performed by computing a mean value indicative of a low frequency component and by canceling that low frequency component in the measured data, because with this approach potentially also information having a physiological origin may be canceled, which is to be avoided.

For this reason, it herein is proposed to compute a fitted curve based on fit values, the fitted curve being, for example, determined by applying a fitting technique, such as a linear regression technique, for example a least squares estimation. Based on the fitted curve, then, the processing data may be corrected in order to compensate for a drift effect in the measured data.

In one embodiment, the step of obtaining the processing data includes: obtaining measured data from the implantable pressure sensor: obtaining a reference value; and deriving the processing data based on the measured data and the reference value. In particular, the processing data may be derived by forming a difference between the measured data and the reference value, wherein the reference value, for example, corresponds to a measured atmospheric pressure value. This is based on the fact that an implantable pressure sensor generally measures an (absolute) internal pressure within the patient, which is subject to the atmospheric pressure around the patient. As the relative pressure within the patient is of primary interest, a reference value corresponding to the atmospheric pressure may be subtracted from the measured data, such that processing data is obtained which is indicative of the internal pressure relative to atmospheric pressure.

As the implantable pressure sensor, in an operative state, is implanted in a patient, the implantable pressure sensor cannot by itself measure the atmospheric pressure outside of the patient. Hence, in one embodiment the reference value corresponding to the atmospheric pressure is measured by an external device external to the implantable pressure sensor, for example a patient device operable outside of the patient and being in communication connection with the implantable pressure sensor. The patient device may, for example, be part of a telemedical monitoring system, the patient device functioning as a relay for relaying data in between the implantable pressure sensor and a remote telemedical monitoring service center, the patient device, for example, having the shape of a mobile communication device, such as a smart phone or a tablet computer.

The external device may be configured in a way, that the reference value could be determined without any drift meaning the method of determining the reference value is either stable in time or is updated on a regular and frequent base.

In one embodiment, the step of deriving the multiplicity of fit values includes: deriving the fit values by filtering the processing data. By means of the filtering in particular high-frequency components may be removed, such that the filtered processing data corresponds to components of the processing data in a lower frequency range, for example below a cut-off frequency. Based on the filtered data, then, the fit values may be determined at regular intervals, such that a number of data points are obtained which are usable to derive the fitted curve by employing a fitting technique, such as a linear regression technique.

For example, in one embodiment the fit values each are derived by computing an average of a predefined multiplicity of samples of the processing data. Each fit value hence is determined by averaging over a predefined multiplicity of samples, for example in between 5 to 100 samples, for example 10 to 30 samples. The fit values hence are determined by averaging the processing data, wherein the fit values are taken at regular intervals of the processing data and are used as input data for the fitting technique to derive the fitted curve.

The implantable pressure sensor may output measured data which correspond to pressure values, averaged, for example, over a short time span, for example a time span between 1 s to 20 s, for example 10 s. Such measured data may be forwarded to the external device at regular intervals or in an event driven manner, wherein at the external device fit values are determined by taking multiple sample values into account and averaging over a multiplicity of sample values corresponding to a prolonged time span, for example multiple hours or even days.

For example, a linear regression technique may be employed to derive the fitted curve based on the fit values. For example, a polynomial regression may be used, for example a quadratic polynomial regression or a cubic polynomial regression. Within the regression the fit values are fitted to a model which is defined by parameters, wherein as a result of the regression the parameters of the model are determined in order such that the fitted curve is defined.

In general, different parametric models may be used to derive the fitted curve, such as polynomial models.

In one embodiment, a parametric function is used as a model for the fitting technique, the parametric function being expressed as

$$f ( t ) = a - b \cdot {e}^{- c \cdot t}$$

where f(t) is the parametric function, and a, b, c are parameters determined by fitting said multiplicity of fit values to the parametric function. In this example, an exponential equation is used as a model, wherein in the course of the fitting to the fit values the parameters of the model are determined such that the fitted curve is defined according to the equation and the specific values for the parameters as derived during the fitting.

In addition, a model for lower frequency components of interference signals may be employed in order to improve a modeling of a drift effect.

Fit values may be determined over the entire lifespan of operation of the implantable pressure sensor in cooperation with an external device in communication connection with the implantable pressure sensor. The correction herein may be applied once a sufficient number of fit values are obtained, wherein the correction may already start in a state of the implantable pressure sensor in which the implantable pressure sensor is not yet implanted in a patient. The earlier the correction starts, the more efficient the correction may be, as a drift effect may be strongest at an initial start of operation of the implantable pressure sensor and may progressively build up from the start of operation of the implantable pressure sensor.

During operation of the implantable pressure sensor, fit values may be determined continuously anew based on the processing data as obtained based on the measured data from the implantable pressure sensor. Based on the new fit values, the correction may be updated, wherein the fitted curve may be determined anew based on the actual set of fit values, e.g., with each new fit value or after a predefined number of new fit values have been obtained.

In one embodiment, the fit values are derived by an external device, for example a patient device, and likewise the fitted curve is determined in the external device for correcting the processing data. As the external device, in comparison to the implantable pressure sensor, may comprise increased computational capabilities and energy resources, data processing may beneficially be carried out by the external device rather than by the implantable pressure sensor. In addition, as the implantable pressure sensor is not able to measure the atmospheric pressure, only the external device can set the measured data in relation to the atmospheric pressure.

In one embodiment, the correction of the processing data takes place by forming a difference between the fitted curve and the processing data to obtain corrected processing data. In particular, the processing data may be corrected by subtracting (sample values obtained according to) the fitted curve from the processing data. As the fitted curve is fitted to fit values which are progressively obtained during operation of the implantable pressure sensor, the fitted curve can be assumed to represent a drift effect, such that by subtracting the fitted curve from the processing data the drift effect is canceled within the processing data, such that the corrected processing data is substantially free of a drift.

For forming the difference between the fitted curve and the processing data, sample values as obtained according to the fitted curve, for example according to a mathematical function representing the fitted curve, are subtracted from the processing data. Hence, mathematically a difference is formed between the curve of the processing data and the fitted curve, such that a drift effect as modeled by the fitted curve is canceled within the processing data.

In one embodiment, the external device may be configured to transmit the corrected processing data to a remote telemedical monitoring service center, such that the corrected data, indicative of the actual, measured relative pressure within the patient, is forwarded to the remote telemedical monitoring service center and may be analyzed by healthcare personnel at the telemedical monitoring service center in order to provide for a telemedical monitoring of the patient.

In another aspect, a system for obtaining data indicative of a pressure in a patient comprises: an implantable pressure sensor configured to obtain measured data indicative of a measured pressure; and a device external to the implantable pressure sensor, the device being configured to obtain the processing data based on the measured data obtained using the implantable pressure sensor: to derive, from said processing data, a multiplicity of fit values: to determine, using said fit values, a fitted curve; and to correct said processing data based on the fitted curve to compensate for a drift effect in said measured data obtained using the implantable pressure sensor.

The advantages and advantageous embodiments described above for the method equally apply also to the system, such that it shall be referred to the above in this respect.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The idea of the present invention shall subsequently be described in more detail with reference to the embodiments as shown in the figures. Herein,

FIG. 1 shows a schematic drawing of an implantable pressure sensor implanted in a patient and in communication with an external patient device;

FIG. 2 shows a schematic drawing of the implantable pressure sensor together with the external patient device; and

FIG. 3 shows curves of processing data, averaged data and fitted data as obtained during a correcting of the processing data.

### DETAILED DESCRIPTION

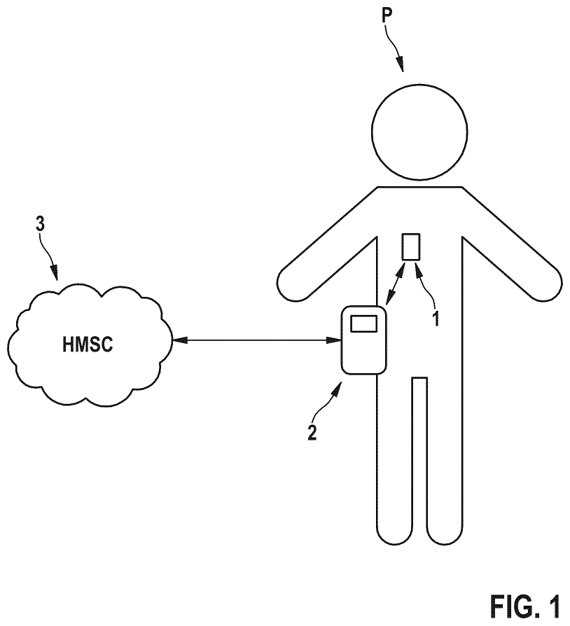

FIG. 1 shows, in a schematic drawing, a general setup of a telemedical monitoring system in which a medical device in the shape of a pressure sensor 1 is implanted in a patient P and is in communication connection with an external device 2 in the shape of a patient device external to the patient. The external device 2 in turn is in communication connection with a telemedical monitoring service center 3 remote from the patient P, the patient device 2 serving as a relay for relaying data in between the pressure sensor 1 implanted in the patient P and the telemedical monitoring service center 3 remote from the patient P.

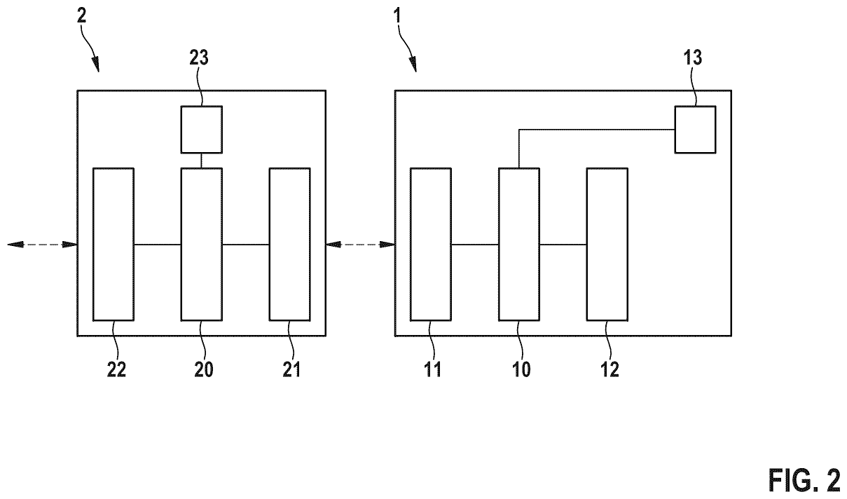

Referring now to FIG. 2, the implantable pressure sensor 1 comprises a processor 10, communication circuitry 11 to establish a (wireless) communication with the external device 2, an energy storage 12 in the shape of a battery, and a sensing device 13 for sensing a pressure internally within the patient. The pressure sensor 1 may, for example, be adapted for placement in the pulmonary artery of the patient, such that by means of the implantable pressure sensor 1 a pressure within the pulmonary artery may be continuously monitored.

The external device 2 in the shape of the patient device comprises a processor 20, communication circuitry 21 for (wirelessly) communicating with the implantable pressure sensor 1, communication circuitry 22 for establishing a (wireless) communication connection with the telemedical monitoring service center 3, and a sensing device 23 for sensing and atmospheric pressure outside of the patient.

The communication circuitry 11, 21 of the pressure sensor 1 and the external device 2 may, for example, be configured to establish a communication by employing a short range communication technique, such as a Bluetooth technique, for example Bluetooth Low Energy (BLE), or a telemetry technique. Communication between the implantable pressure sensor 1 and the external device 2 generally is established in a wireless fashion.

The communication circuitry 22 of the external device 2, in contrast, may be adapted to establish a communication connection to the telemedical monitoring service center 3 employing a public communication technology, for example a mobile communication technology, such as a 3G, 4G, or 5G technique.

By means of the sensing device 13 of the implantable pressure sensor 1, data indicative of a pressure in the vicinity of the implantable pressure sensor 1 is continuously measured during the operational life span of the pressure sensor 1. The measured data is transmitted from the implantable pressure sensor 1 towards the external device 2, where the measured data is processed and is forwarded to the telemedical monitoring service center 3. At the telemedical monitoring service center 3, the data may be analyzed, wherein the telemedical monitoring service center 3 may provide a web interface allowing a user to access the data remotely from the patient P.

In one embodiment, the measured data obtained from the implantable pressure sensor 1 is forwarded to the external device 2 and is set, during a processing within the processor 20 of the external device 2, in relation to a reference value corresponding to the atmospheric pressure as measured by the sensing device 23 of the external device 2. Hence, processing data is obtained, which is indicative of a relative pressure within the patient P. The measurement of the atmospheric pressure by sensing device 23 of the external device 2 is either stable in time or calibrated on a regular and frequent base. Consequently there is no need to compensate or calculated any drift originating in the reference value.

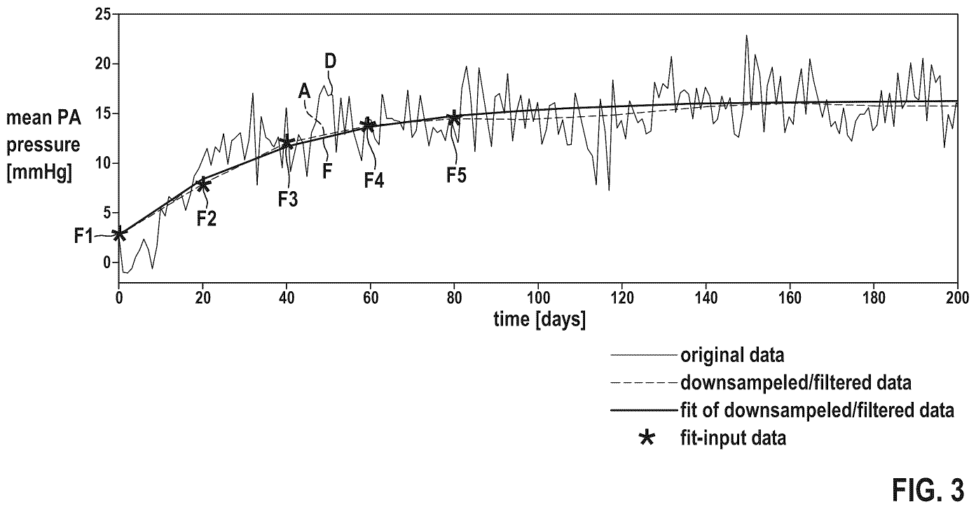

Processing data D in an example is illustrated in FIG. 3, the processing data D exhibiting a progressive increase due to lower frequency components within the processing data D. Positive and negative peaks corresponding to higher frequency components of the processing data D are indicative of pressure variations within the patient P.

A pressure sensor 1 could exhibit a drift in rare cases, which may be due to an aging of the pressure sensor 1 and due to strains and stresses acting onto the pressure sensor 1. A drift effect may progressively change measured data, in that it may progressively, for example, cause an increase of the measured data, which however does not correspond to an increase in the actual pressure within the patient. Hence, in order to improve the accuracy of the measured data obtained from the pressure sensor 1, it is desirous to compensate for a drift effect, causing a progressive, slow change in the measured data due to effects other than actual pressure changes within the patient.

Referring again to the example of FIG. 3, it generally can be assumed that lower frequency components in the processing data D are substantially due to a drift effect. Hence, by modeling the lower frequency components and by canceling the lower frequency components within the processing data D, a drift effect may be compensated for, such that a more accurate pressure reading becomes possible.

For compensating for a drift effect, it herein is proposed to determine fit values F1, F2, F3, F4, F5 from the processing data D. The fit values F1-F5 herein are determined, in one embodiment, by averaging the processing data D to obtain averaged data A, the fit values F1-F5 being taken at regular intervals, for example at every 20 samples, of the averaged data A, as illustrated in FIG. 3.

Based on the fit values F1-F5, then, a fitted curve F is determined, for example by employing a linear regression technique for fitting a parametric model to the fit values F1-F5 to obtain the fitted curve F.

For example, a polynomial regression may be employed, in which the fit values F1-F5 are fitted to a polynomial model, for example a quadratic model or a cubic model. This however is not limiting for the present invention. Generally, any model which may reliably model a progressive change in sensory data may be used, wherein the model may be adapted during operation of the implantable pressure sensor 1.

In a specific example, the fitting employs a parametric function according to

f(t)=a−b·e^{-C·t}
where f(t) is the parametric function, and a, b, c are parameters determined by fitting said multiplicity of fit values F1-F5 to the parametric function. During the fitting to the fit values F1-F5, the parameters a, b, c are determined, such that a fitted curve as mathematically described by the parametric function is obtained.

In addition, for example lower frequency components of an interference signal may be modeled in order to improve a modeling of a drift effect.

Once a fitted curve F is obtained by fitting a suitable model to the fit values F1-F5, the processing data D may be corrected by subtracting sample values obtained according to the fitted curve F from the processing data D, such that a progressive change in the processing data D due to a drift effect as modeled by the fitted model is canceled within the processing data D, the corrected processing data hence being substantially drift free and accurately indicating a measured pressure within the patient.

Correction may start at the initial start of operation of the implantable pressure sensor 1. The correction herein may be applied once a sufficient number of fit values F1-F5 are obtained, wherein in principle any number (equal to or larger than a predetermined minimum number) of fit values F1-F5 may be used as input for the model in order to, e.g., determine parameters of a parametric function mathematically describing the model.

The correction herein may start already prior to implanting the implantable pressure sensor 1 into a patient. Generally, the sooner the correction is started in order to compensate for a drift effect, the more accurate the compensation may be, as a change in the measured data due to a drift effect may potentially be largest, as illustrated in FIG. 3, at the beginning of operation of the implantable pressure sensor 1.

During the continuous operation of the implantable pressure sensor 1, fit values are continuously determined anew. With each new fit value or after a predefined number of new fit values are obtained, the fitted curve F may be determined anew by fitting the model to the now available set of fit values. The model hence may be continuously updated and adapted during operation of the implantable pressure sensor 1, such that a drift effect may be accurately modelled over the life span of the implantable pressure sensor 1.

The correction generally takes place in the external device 2, which may be a patient device in the vicinity of the patient. The external device 2 herein, after employing the correction, forwards the corrected processing data to the telemedical monitoring service center 3, the corrected data being indicative of the (relative) internal pressure within the patient as measured by the pressure sensor 1, a drift effect being compensated within the corrected data.

In another embodiment, the processing of the data for correcting a drift effect takes place only in the telemedical monitoring service center 3, such that the external device 2 forwards the (non-corrected) processing data to the telemedical monitoring service center 3, which applies a suitable correction to compensate for a drift effect.

The idea of the present invention is not limited to the embodiments described above, but may be implemented in an entirely different fashion.

The implantable pressure sensor may be adapted for implantation into the pulmonary artery, wherein the present invention is also applicable to other implantable pressure sensor devices to be implanted at other locations in a patient.

The implantable pressure sensor may also be part of an implantable medical device, for example an implantable stimulation device, such as a pacemaker device or a defibrillation device.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 Implantable pressure sensor
10 Processor
11 Communication circuitry
12 Energy storage (battery)
13 Sensing device
2 patient device (patient device)
20 Processor
21, 22 Communication circuitry
23 Sensing device
3 Telemedical monitoring service center
A Averaged data
F Fitted curve
F1-F5 Fit values
D Processing data
P Patient

1. A method for correcting a drift effect in measured data obtained using an implantable pressure sensor, the method comprising:
obtaining processing data, based on measured data indicative of a measured pressure obtained using the implantable pressure sensor;
deriving, from said processing data, a multiplicity of fit values;
determining, using said fit values, a fitted curve; and
correcting said processing data based on the fitted curve to compensate for a drift effect in said measured data obtained using the implantable pressure sensor.

2. The method according to claim 1, wherein said step of obtaining processing data includes:
obtaining measured data from the implantable pressure sensor;
obtaining a reference value; and
deriving the processing data based on the measured data and the reference value.

3. The method according to claim 2, wherein the processing data are derived by forming a difference between said measured data and the reference value.

4. The method according to claim 2, wherein said reference value is indicative of the atmospheric pressure.

5. The method according to claim 2, wherein said reference value is measured by a patient device operable outside of a patient and being in communication connection with said implantable pressure sensor.

6. The method according to claim 1, wherein said step of deriving a multiplicity of fit values includes: deriving said fit values by filtering the processing data.

7. The method according to claim 1, wherein said step of deriving a multiplicity of fit values includes: deriving each of said fit values by computing an average of a predefined multiplicity of samples of said processing data.

8. The method according to claim 1, wherein said step of determining a fitted curve includes: fitting said multiplicity of fit values to a parametric function.

9. The method according to claim 8, wherein said parametric function is mathematically expressed as:
$$f ( t ) = a - b \cdot {e}^{- c \cdot t}$$
where f(t) is the parametric function, and a, b, c are parameters determined by fitting said multiplicity of fit values to the parametric function.

10. The method according to claim 1, wherein fit values are determined repeatedly anew during operation of the implantable pressure sensor.

11. The method according to claim 10, wherein said steps of determining a fitted curve and correcting said processing data are repeated at the availability of a new fit value.

12. The method according to claim 1, wherein said steps of deriving a multiplicity of fit values, determining a fitted curve and correcting said processing data are carried out in a device external to the implantable pressure sensor.

13. The method according to claim 1, wherein said step of correcting said processing data includes: forming a difference between correction the fitted curve and the processing data to obtain corrected processing data.

14. The method according to claim 1, wherein an additional step of transmitting corrected processing data, obtained as a result of said step of correcting said processing data, to a telemedical monitoring service center external to the implantable pressure sensor.

15. A system for obtaining data indicative of a pressure in a patient, comprising:
an implantable pressure sensor configured to obtain measured data indicative of a measured pressure; and
a device external to the implantable pressure sensor, the device being configured
to obtain processing data-based on the measured data obtained using the implantable pressure sensor;
to derive, from said processing data, a multiplicity of fit values;
to determine, using said fit values, a fitted curve; and
to correct said processing data based on the fitted curve to compensate for a drift effect in said measured data obtained using the implantable pressure sensor.
